(19) United States
(12) Patent Application Publication
(10) Pub. No.: US 2020/0078407 A1
(43) Pub. Date: Mar. 12, 2020

(54) BLOOD PLASMA-CONTAINING COMPOSITIONS

(71) Applicant: Cellphire, Inc., Rockville, MD (US)

(72) Inventors: Biplob Bhattacharya, Towson, MD (US); Anne S. Hale, Placitas, NM (US); Rafael Jorda, Bethesda, MD (US); Glen Michael Fitzpartick, North Potomac, MD (US)

(21) Appl. No.: 16/567,737

(22) Filed: Sep. 11, 2019

Related U.S. Application Data
(60) Provisional application No. 62/729,830, filed on Sep. 11, 2018.

(51) Int. Cl. A61K 35/19; A61K 35/16; A61K 31/715; A61K 31/205; A61K 31/14; A61K 31/685; A61K 31/7016; A61K 9/19; A61P 7/04

(52) U.S. Cl. CPC A61K 35/19; A61K 35/16; A61K 31/715; A61K 31/205; A61P 7/04; A61K 31/685; A61K 31/7016; A61K 9/19; A61K 31/14

(57) ABSTRACT

Provided herein is a composition of matter comprising dried blood plasma substantially free of blood cell components; and a) a resuscitative mixture comprising at least one saccharide, at least one salt, and at least one high molecular weight, non-ionic, hydrophilic polymer, b) platelets or platelet-derived material, or c) both a) and b).

### CROSS-REFERENCE TO RELATED APPLICATIONS

This application claims priority to U.S. Provisional Application No. 62/729,830, filed Sep. 11, 2018, which is incorporated by reference in its entirety herein.

### BACKGROUND

### Technical Field

The document relates to the field of medicine. Provided herein are compositions comprising one or more of platelets, plasma, or a mixture comprising at least one salt and at least one high molecular weight, non-ionic, hydrophilic polymer, as well as methods for treating hypovolemia with such compositions.

### SUMMARY

Provided herein are compositions that include three components: a) one or more of platelets or platelet-derived material; b) plasma; and c) a mixture comprising at least one salt and at least one high molecular weight, non-ionic, hydrophilic polymer. In some embodiments of the composition provided herein, the mixture further comprises a buffer and/or at least one saccharide.

In some embodiments, at least one of

(a) the platelets or platelet-derived material,

or

(b) the plasma,

is dried.

In some embodiments, at least one of

(a) the platelets or platelet-derived material,

or

(b) the mixture comprising at least one salt and at least one high molecular weight, non-ionic, hydrophilic polymer,

is dried.

In some embodiments, at least one of

(a) the plasma,

or

(b) the mixture comprising at least one salt and at least one high molecular weight, non-ionic, hydrophilic polymer,

is dried.

In some embodiments, at least one of

(a) the platelets or platelet-derived material,

(b) the plasma,

or

(c) the mixture comprising at least one salt and at least one high molecular weight, non-ionic, hydrophilic polymer,

is dried.

Additionally, some embodiments of the compositions provided herein comprise one or more of platelets or platelet-derived material, plasma, or a mixture comprising at least one salt and at least one high molecular weight, non-ionic, hydrophilic polymer. In some embodiments, the compositions provided herein comprise two or more of platelets or platelet-derived material, plasma, or a mixture comprising at least one salt and at least one high molecular weight, non-ionic, hydrophilic polymer. In some embodiments, the compositions provided herein comprise platelets or platelet-derived material, plasma, and a mixture comprising at least one salt and at least one high molecular weight, non-ionic, hydrophilic polymer. In some embodiments, the composition provided herein includes the mixture, which further comprises a buffer and/or at least one saccharide.

In some embodiments, at least one of

(a) the platelets or platelet-derived material,

or

(b) the plasma,

is dried.

In some embodiments, at least one of

(a) the platelets or platelet-derived material,

or

(b) the mixture comprising at least one salt and at least one high molecular weight, non-ionic, hydrophilic polymer,

is dried.

In some embodiments, at least one of

(a) the plasma,

or

(b) the mixture comprising at least one salt and at least one high molecular weight, non-ionic, hydrophilic polymer,

is dried.

In some embodiments, at least one of

(a) the platelets or platelet-derived material,

(b) the plasma,

or

(c) the mixture comprising at least one salt and at least one high molecular weight, non-ionic, hydrophilic polymer,

is dried.

In some embodiments, the compositions comprising one or more of platelets or platelet-derived material, plasma, or a mixture comprising at least one salt and at least one high molecular weight, non-ionic, hydrophilic polymer, are infusible compositions, such as blood-infusible compositions. In some embodiments, the compositions provided herein are injectable compositions.

In some embodiments, the compositions comprising one or more of platelets or platelet-derived material, plasma, or a mixture comprising at least one salt and at least one high molecular weight, non-ionic, hydrophilic polymer, further comprise red blood cells or red blood cell substitutes. Thus, in some embodiments, provided herein is a composition of matter comprising:

red blood cells or red blood cell substitutes; and
a) a resuscitative mixture comprising at least one salt and at least one high molecular weight, non-ionic, hydrophilic polymer,
b) platelets or platelet-derived material,

or
c) both a) and b).

In some embodiments, provided herein is a composition of matter comprising:

dried blood plasma substantially free of blood cell components;

red blood cells or red blood cell substitutes; and

and
a) a resuscitative mixture comprising at least one salt and at least one high molecular weight, non-ionic, hydrophilic polymer,
b) platelets or platelet-derived material,

or
c) both a) and b).

In some embodiments, provided herein is a composition of matter comprising:

red blood cells or red blood cell substitutes; and
a) a resuscitative mixture comprising at least one salt and at least one high molecular weight, non-ionic, hydrophilic polymer,

and
b) platelets or platelet-derived material,

In some embodiments, provided herein is a composition of matter comprising:

red blood cells or red blood cell substitutes; and
a resuscitative mixture comprising at least one salt and at least one high molecular weight, non-ionic, hydrophilic polymer.

In some embodiments, provided herein is a composition of matter comprising:

dried blood plasma substantially free of blood cell components;

red blood cells or red blood cell substitutes; and
a) a resuscitative mixture comprising at least one salt and at least one high molecular weight, non-ionic, hydrophilic polymer,

and
b) platelets or platelet-derived material,

In some embodiments, provided herein is a composition of matter comprising:

dried blood plasma substantially free of blood cell components;

red blood cells or red blood cell substitutes; and
a resuscitative mixture comprising at least one salt and at least one high molecular weight, non-ionic, hydrophilic polymer.

In some embodiments, provided herein is a composition of matter comprising:

dried blood plasma substantially free of blood cell components;
a) a resuscitative mixture comprising at least one salt and at least one high molecular weight, non-ionic, hydrophilic polymer,

and
b) platelets or platelet-derived material,

In some embodiments, provided herein is a composition of matter comprising:

dried blood plasma substantially free of blood cell components; and
a resuscitative mixture comprising at least one salt and at least one high molecular weight, non-ionic, hydrophilic polymer.

In some embodiments, provided herein is a method of treating hypovolemia in a subject, comprising administering to the subject a composition as disclosed herein.

In some embodiments, the composition is present in an effective amount. An “effective amount” of a composition is an amount of the composition that is effective in treating a disease or condition or disorder as recited herein, such as hypovolemia or such as blood loss, or that is effective in controlling bleeding.

In some embodiments, the composition present in an effective amount is a composition that comprises an effective amount of platelets or platelet-derived material (e.g., thrombosomes). An effective amount of platelets or platelet-derived material is any appropriate dosage of a composition comprising platelets or platelet-derived material as described herein that can be administered to the subject. For example, in some embodiments, an effective amount is about 1.0×10⁷ particles to about 1.0×10¹⁰ particles, such as about 1.6×10⁷ particles (e.g., thrombosomes)/kg to about 1.0×10¹⁰ particles/kg (e.g., about 1.6×10⁷ to about 5.1×10⁹ particles/kg, about 1.6×10⁷ to about 3.0×10⁹ particles/kg, about 1.6×10⁷ to about 1.0×10⁹ particles/kg, about 1.6×10⁷ to about 5.0×10⁸ particles/kg, about 1.6×10⁷ to about 1.0×10⁸ particles/kg, about 1.6×10⁷ to about 5.0×10⁷ particles/kg, about 5.0×10⁷ to about 1.0×10⁸ particles/kg, about 1.0×10⁸ to about 5.0×10⁸ particles/kg, about 5.0×10⁸ to about 1.0×10⁹ particles/kg, about 1.0×10⁹ to about 5.0×10⁹ particles/kg, or about 5.0×10⁹ to about 1.0×10¹⁰ particles/kg).

In some embodiments, the composition present in an effective amount is a composition that comprises an effective amount of one or more components of the resuscitative mixture comprising at least one salt and at least one high molecular weight, non-ionic, hydrophilic polymer. Examples of effective amounts of components of the mixture, such as effective amounts of at least one salt, effective amounts of at least one high molecular weight, non-ionic, hydrophilic polymer, effective amounts of a buffer, and effective amounts of at least one saccharide, are disclosed herein. In some embodiments, the effective amount of a resuscitative mixture comprising at least one salt and at least one high molecular weight, non-ionic, hydrophilic polymer is any appropriate dosage of a composition comprising the resuscitative mixture comprising at least one salt and at least one high molecular weight, non-ionic, hydrophilic polymer as described herein that can be administered to the subject. For example, in some embodiments, an effective amount of the resuscitative mixture comprising at least one salt and at least one high molecular weight, non-ionic, hydrophilic polymer is about 1 mL/kg, 2 mL/kg, 3 mL/kg, 4 mL/kg, 5 mL/kg, 6 mL/kg, 7 mL/kg, 8 mL/kg, 9 mL/kg, or 10 mL/kg. In some embodiments, the composition present in an effective amount is a composition that comprises an effective amount of the resuscitative mixture in any appropriate dosage to form a composition that is a colloid fluid.

In some embodiments, provided herein is a method of treating blood loss and/or control bleeding in a subject, comprising administering to the subject a composition as disclosed herein. The method of treating blood loss and/or control bleeding can include using or administering platelets or platelet-derived material, plasma, and/or a mixture comprising at least one salt and at least one high molecular weight, non-ionic, hydrophilic polymer. In some embodiments, the method of treating blood loss comprises using or administering red blood cells. In some embodiments, the method of treating blood loss does not comprise using or administering red blood cells.

In some embodiments, provided herein are compositions of matter, methods of making them, and methods of using them for expanding blood volume in a subject having reduced blood volume. Additionally, provided herein are kits for making, supplying, and/or storing the compositions of matter.

In some embodiments, provided herein are compositions of matter, methods of making them, and methods of using them for expanding blood volume in a human subject. In some embodiments, the subject may be a non-human animal.

In some embodiments, provided herein are methods of using compositions of matter provided herein that includes infusing or injecting the compositions of matter into the subject's blood. For example, in some embodiments, the compositions of matter are infused or injected into a subject at a desirable infusion rate, e.g., 1.0 mL/min.

In some embodiments, the compositions of matter provided herein may provide synergistically superior blood volume expansive and resuscitative properties that yield hemodynamic and/or hemostatic benefits. In some embodiments, the compositions of matter provided herein may provide superior blood volume expansion and\or resuscitation as compared to blood plasma alone or as compared to the additional component(s) alone. With reference to exemplary compositions of matter provided herein, the combination of dried blood plasma with a resuscitative mixture may provide a blood resuscitative fluid that synergistic in its effect on blood volume expansion.

In some embodiments, the compositions of matter provided herein comprise a mixture comprising at least one salt and at least one high molecular weight, non-ionic, hydrophilic polymer, wherein the mixture is a balanced synthetic colloid resuscitation fluid. The colloids present in some embodiments of the compositions of matter provided herein can act as plasma volume expanders in the treatment of hypovolemic and/or hemorrhagic shock. The colloids can facilitate plasma volume expansion by including large molecules that are retained in the blood vessels and do not readily cross capillary walls. In some embodiments, the compositions of matter provided herein comprise a crystalloid resuscitation fluid. A crystalloid solution generally has a higher concentration of electrolytes than body plasma.

In one embodiment, the compositions of matter comprise plasma such as dried blood plasma and one or more additional components. In some embodiments, the compositions of matter may provide superior blood volume expansion or resuscitation as compared to blood plasma alone or as compared to the additional component(s) alone. In some embodiments, the additional component(s) can be a resuscitative composition such as a mixture comprising at least one salt and at least one high molecular weight, non-ionic, hydrophilic polymer; platelets or platelet-derived material such as dried platelets or platelet-derived material; or both. The compositions of matter can be in dry form or liquid form. In some embodiments, the blood plasma of the composition of matter is in dry form and at least one of the additional components is in liquid form. As used herein, “in liquid form” refers to a solution or suspension of a component or components in a liquid, or a mixture of the component or components with a liquid, such as a solution or suspension in an aqueous liquid, or a mixture with an aqueous liquid, such as an aqueous solution. In some embodiments, the composition of matter may be in liquid form in methods of using the composition for treatment of subjects in need of blood volume expansion or resuscitation.

As used herein “wt/v” refers to weight of a component over volume of a composition comprising the component, and “v/v” refers to volume of a component over volume of a composition comprising the component.

In another embodiment, methods of making the compositions of matter herein are provided. The methods of making can vary according to the desired formulation chosen by the practitioner. In some embodiments, the plasma, such as dried blood plasma is substantially free of blood cell components—that is, red blood cells, white cells, platelets, and platelet-derived material—where “substantially free” herein means that as much of the blood cell components are separated from the blood plasma of the animal (e.g., human or other mammalian subjects) as can be achieved using standard processes known in the art to separate blood cell components from plasma. In some embodiments, plasma, such as dried blood plasma substantially free of blood cell components contains fewer than about 10,000 platelets per microliter (μL) (e.g., fewer than about 10,000 platelets/μL, fewer than about 8,000 platelets/μL, fewer than about 6,000 platelets/μL, fewer than about 4,000 platelets/μL, fewer than about 2,000 platelets/μL, or fewer than about 1,000 platelets/μL) when the plasma is in a liquid form, e.g., when the plasma has been rehydrated. As used herein, a unit recitation that includes a unit of volume (e.g., μL) includes compositions that are in liquid form when in an original or rehydrated state as well as compositions that are in dried form and that provide the desired amount of platelets/μL upon rehydration with an appropriate amount of liquid. By way of example, a recitation of 4,000 platelets/μL may be used to indicate a composition comprising 4,000,000 platelets that is rehydrated to provide a volume of 1 mL. A limit (or a range) provided herein for the “platelets” encompasses a limit (or a range) of platelets in platelet-containing compositions as well as a limit (or a range) of platelets or particles in compositions containing platelet-derived materials. For example, a limit of 10,000 platelets/μL of a platelet-containing composition also encompasses 10,000 platelet or particles/μL of a composition containing platelet-derived materials. In some embodiments, a concentration of platelets is no greater than 7.0×10¹¹/mL for patient safety reasons. Additional components can be provided in dry form or liquid form. In exemplary embodiments, methods of making a composition of matter include making a dried blood plasma composition, separately making a dried colloidal blood volume resuscitation mixture, adding an aqueous liquid to either the dried blood plasma composition or the dried colloidal blood volume resuscitation mixture, and combining the composition and the mixture to make a composition of matter in liquid form. In other exemplary embodiments, blood plasma can be combined with the components of a colloidal blood volume resuscitation mixture, and the resulting composition dried.

In some embodiments, the dried composition can be rehydrated with an aqueous liquid.

Additional or alternative compositions as supplements or replacements for the colloidal blood volume resuscitation mixture can be included in the method of making a composition of matter as disclosed herein.

In some embodiments, the methods of making a composition as disclosed herein comprise a lyophilization step. In some embodiments, the methods of making a composition do not comprise a pathogen reduction step prior to the lyophilization step.

In some embodiments, provided herein is a composition of matter comprising:

dried blood plasma; and
a) a resuscitative mixture comprising at least one salt and at least one high molecular weight, non-ionic, hydrophilic polymer,
b) platelets or platelet-derived material,

or
c) both a) and b).

In some embodiments, provided herein is a composition of matter comprising:

dried blood plasma; and
a) red blood cells or red blood cell substitutes,
b) a resuscitative mixture comprising at least one salt and at least one high molecular weight, non-ionic, hydrophilic polymer, and
c) platelets or platelet-derived material,

or
d) each of a) and b) and c).

In some embodiments, provided herein is a composition of matter comprising:

dried blood plasma substantially free of blood cell components; and
a) a resuscitative mixture comprising at least one salt and at least one high molecular weight, non-ionic, hydrophilic polymer,
b) platelets or platelet-derived material,

or
c) both a) and b).

In some embodiments, provided herein is a composition of matter comprising: dried blood plasma substantially free of blood cell components; and
a) red blood cells or red blood cell substitutes,
b) a resuscitative mixture comprising at least one salt and at least one high molecular weight, non-ionic, hydrophilic polymer,
c) platelets or platelet-derived material,

or
c) each of a) and b) and c).

In some embodiments of compositions comprising two or more of platelets (or platelet-derived material), plasma, or a mixture comprising at least one salt and at least one high molecular weight, non-ionic, hydrophilic polymer,

the plasma is present in an amount from about 30% to about 80% wt/v; and
the platelets (or platelet-derived material) are present in a concentration of about 10,000 platelets/μL to about 2,000,000 platelets/μL.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 40% to about 70% wt/v; and the platelets (or platelet-derived material) are present in a concentration of about 100,000 platelets/μL to about 1,000,000 platelets/μL.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 30% to about 80% wt/v; and
the mixture comprising at least one salt and at least one high molecular weight, non-ionic, hydrophilic polymer, is present in an amount from about 20% to about 70% wt/v

In some embodiments of compositions comprising two or more of platelets (or platelet-derived material), plasma, or a mixture comprising at least one salt and at least one high molecular weight, non-ionic, hydrophilic polymer,

the plasma is present in an amount from about 40% to about 70% wt/v; and
the mixture comprising at least one salt and at least one high molecular weight, non-ionic, hydrophilic polymer, is present in an amount from about 30% to about 60% wt/v.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 30% to about 80% wt/v; and the platelets (or platelet-derived material) are present in a concentration of about 10,000 platelets/μL to about 2,000,000 platelets/μL.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 40% to about 70% wt/v; and
the platelets (or platelet-derived material) are present in a concentration of about 100,000 platelets/μL to about 1,000,000 platelets/μL.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 30% to about 80% wt/v; and
the mixture comprising at least one salt and at least one high molecular weight, non-ionic, hydrophilic polymer, is present in an amount from about 20% to about 70% wt/v

In some embodiments of compositions comprising two or more of platelets (or platelet-derived material), plasma, or a mixture comprising at least one salt and at least one high molecular weight, non-ionic, hydrophilic polymer,

the plasma is present in an amount from about 40% to about 70% wt/v; and
the mixture comprising at least one salt and at least one high molecular weight, non-ionic, hydrophilic polymer, is present in an amount from about 30% to about 60% wt/v.

In some embodiments of the composition, the amount of plasma and the amount of platelets are in a ratio as disclosed in Holcomb et. al., Crit Care Clin. 2017; 33(1): 15-36, incorporated by reference herein in its entirety.

In some embodiments, any one of the compositions provided herein comprises red blood cells or red blood cell substitutes, wherein the red blood cells or red blood cell substitutes are present in an amount from about 2 to about 8 million units per microliter. In some embodiments, the red blood cells or red blood cell substitutes are present in an amount from about 3 to about 7 million units per microliter. In some embodiments, the red blood cells or red blood cell substitutes are present in an amount from about 4 to about 6 million units per microliter. In some embodiments, the red blood cells or red blood cell substitutes are present in an amount from about 4 to about 5 million units per microliter or from about 5 to about 6 million units per microliter.

In some embodiments, at least one of a), b), or c), is freeze-dried. As used herein, “freeze-dried” refers to a drying method achieved using lyophilization. As such, terms such as “freeze-dried” and “lyophilized” can be used interchangeably in this document.

In some embodiments, a) further comprises at least one saccharide, a buffer, or both at least one saccharide and a buffer.

In some embodiments, a “platelet-derived” material is a material derived from platelets in the manner disclosed in U.S. Pat. No. 8,486,617, incorporated by reference herein in its entirety.

In some embodiments, the platelet-derived material comprises (i), thrombosomes, (ii) microparticles such as microparticles formed by breaking off from platelets, or (iii) both (i) and (ii).

As used herein, “thrombosomes” (sometimes also herein called “Tsomes” or “Ts”) are platelet derivatives that have been treated with an incubating agent (e.g., any of the incubating agents described herein, such as the saccharides described herein) and lyopreserved (such as freeze-dried). In some cases, thrombosomes can be prepared from pooled platelets. Thrombosomes can have a shelf life of 2-3 years in dry form at ambient temperature and can be rehydrated with sterile water within minutes for immediate infusion. One example of thrombosomes are THROMBOSOMES®, which are in clinical trials for the treatment of acute hemorrhage in thrombocytopenic patients.

In some embodiments, a composition of matter as disclosed herein further comprises an aqueous liquid. In some embodiments, the aqueous liquid is water or saline.

In some embodiments, a resuscitative mixture as disclosed herein further comprises an aqueous liquid. In some embodiments, the aqueous liquid is water or saline.

In some embodiments of the compositions of matter as disclosed herein, following rehydration, the platelets, platelet-derived material, or both, further contain an aqueous liquid. In some embodiments, the aqueous liquid is plasma. In some embodiments, the aqueous liquid is a mixture comprising at least one salt and at least one high molecular weight, non-ionic, hydrophilic polymer.

In some embodiments, the dried platelets, platelet-derived material, or both, are freeze-dried, spray-dried, or freeze spray-dried.

In some embodiments, a composition of matter as disclosed herein does not comprise trehalose.

In some embodiments of the compositions of matter as disclosed herein, the dried blood plasma, when rehydrated with an aqueous liquid, has coagulation factor levels showing all individual factors (e.g., Factors VII, VIII and IX) associated with blood clotting at 40 international units (IU) or greater.

In some embodiments, the dried blood plasma, when rehydrated with an aqueous liquid, has coagulation factor that include only Factors VII, VIII and IX.

In some embodiments, the dried blood plasma, when rehydrated with an aqueous liquid, has coagulation factor levels showing individual factors VII, VIII and IX associated with blood clotting at 40 international units (IU) or greater.

In some embodiments, provided herein is a composition of matter comprising: blood plasma rehydrated with an aqueous liquid and substantially free of blood cell components; and
a) a resuscitative mixture comprising at least one saccharide, at least one salt and at least one high molecular weight, non-ionic, hydrophilic polymer,
b) platelets or platelet-derived material,

or
c) both a) and b);

wherein the composition of matter comprises sufficient blood plasma, saccharide(s), salt(s), and high molecular weight, non-ionic, hydrophilic polymer(s) to provide a resuscitative effect when administered to a subject in need of blood resuscitation.

In some embodiments, the composition of matter comprises one or more components to provide a resuscitative effect when administered to a subject in need of blood resuscitation. A “resuscitative effect” is restoring a blood pressure (e.g., mean arterial pressure) and/or blood volume of the patient such that blood vessels do not collapse and blood reaches major organs within the patient. In some embodiments, the resuscitative effect comprises restoring a mean arterial pressure (MAP) to at least 50 mmHg (e.g., to at least 60 mmHg, to at least 70 mmHg, to at least 80 mmHg, to at least 90 mmHg, or to at least 100 mmHg). In some embodiments, the resuscitative effect comprises restoring a mean arterial pressure (MAP) to no more than 100 mmHg (e.g., to no more than 90 mmHg, to no more than 80 mmHg, to no more than 70 mmHg, or to no more than 60 mmHg). In some embodiments, the resuscitative effect comprises restoring a mean arterial pressure (MAP) from about 60 mmHg to about 100 mmHg, or from about 70 mmHg to about 90 mmHg.

In some embodiments, provided herein is a composition of matter comprising:

blood plasma rehydrated with an aqueous liquid, and
a) red blood cells or red blood cell substitutes,
b) a resuscitative mixture comprising at least one saccharide, at least one salt and at least one high molecular weight, non-ionic, hydrophilic polymer,
c) platelets or platelet-derived material,

or
d) each of a) and b) and c);

wherein the composition of matter comprises sufficient blood plasma, saccharide(s), salt(s), and high molecular weight, non-ionic, hydrophilic polymer(s) to provide a resuscitative effect when administered to a subject in need of blood resuscitation.

In some embodiments of the composition of matter comprising blood plasma rehydrated with an aqueous liquid (e.g., the embodiments described above), the blood plasma has coagulation factor levels showing all individual factors associated with blood clotting at 40 international units (IU) or greater.

In some embodiments of the compositions of matter disclosed herein, the at least one high molecular weight, non-ionic, hydrophilic polymer of the resuscitative mixture is a copolymer of sucrose and epichlorohydrin. In some more particular embodiments, the copolymer is polysucrose.

In some embodiments of the compositions of matter disclosed herein, the composition of matter is isotonic.

In some embodiments, a composition of matter as disclosed herein further comprises an additive for preservation of coagulation function. In some embodiments, the additive for preservation of coagulation function L-Carnitine, Propionyl L-Carnitine, Taurine, Glycerophosphocholine (GCP) or Trehalose. In some embodiments, the additive for preservation of coagulation function comprises taurine.

In some embodiments, provided herein is a method of manufacturing dried blood plasma substantially free of blood cell components, comprising:
a) mixing a plurality of plasma samples to form a first mixture;

and
b) lyophilizing or spray-drying the first mixture to form the dried blood plasma substantially free of blood cell components.

In some embodiments, the method of manufacturing further comprises rehydrating the dried blood plasma with an aqueous liquid, to form a hydrated blood plasma.

Methods of using the compositions of matter are also provided herein. The methods of using typically relate to methods of treatment of a subject in need of blood volume expansion or resuscitation. However, it is to be understood that the methods of using the compositions of matter can also be prophylactic methods for treatment of subjects that are about to undergo a procedure where substantial loss of blood is expected or is often seen. For example, hemorrhage remains a major cause of preventable death following both civilian and military trauma.

Hemorrhage accounts for up to 40% of trauma-related deaths, in which most hemorrhage-related deaths occur in the first 6 hours after injury. The goals of resuscitation in the face of hemorrhagic shock are restoring end-organ perfusion and maintaining tissue oxygenation while attempting definitive control of bleeding. It is to be understood that the methods of using the compositions of matter can be in vitro or in vivo methods or diagnostic methods. In general, the methods of using the compositions of matter for treatment of subjects comprise rehydrating the components of the composition of matter (e.g., dried blood plasma, dried platelets or platelet-derived material) with an aqueous liquid, providing appropriate conditions to achieve a homogeneous mixture, and administering the mixture to a subject in need thereof. Similarly, for research or diagnostic methods, the methods comprise rehydrating the components of the composition of matter (e.g., dried blood plasma, dried platelets or dried platelet-derived material) with an aqueous liquid and providing appropriate conditions to achieve a homogeneous mixture. The homogeneous mixture can then be used in research or diagnostic methods

Thus, in some embodiments, provided herein is a method of blood expanding and/or resuscitating in a subject, the method comprising:

rehydrating at least one component of a composition of matter as disclosed herein to obtain a rehydrated composition; and

administering the rehydrated composition to the subject.

In some more particular embodiments, the rehydrated composition is a homogenous mixture.

In some embodiments, kits containing the components of compositions of matter provided herein. As used in this document, a kit is a package that provides some or all of the components of the composition of matter provided herein. The package is suitable for shipping, delivering, etc. the composition components. The package can also be suitable for storage of the composition components until needed for use. Kits for shipping and storage of blood components are described in more detail below.

### BRIEF DESCRIPTION OF THE DRAWINGS

FIG. 1 shows plots of Mean Arterial Pressure (MAP) vs. time for various compositions (t=baseline is the time where an initial mean blood pressure of the animal is taken; t=0 is the time where bleeding is induced (i.e., 40% blood volume is drawn from the subject); t=10 is the time where an intravenous test sample is infused).

FIG. 2 shows plots of % change of Mean Arterial Pressure from baseline vs. time for various compositions (t=baseline is the time where an initial MAP of the animal is taken; t=0 is the time where bleeding is induced; t=10 is the time where an intravenous test sample is infused).

FIG. 3 shows plots of % change of Mean Arterial Pressure from baseline vs. time for various compositions (t=baseline is the time where an initial MAP of the animal is taken; t=0 is the time where bleeding is induced; t=10 is the time where an intravenous test sample is infused).

FIG. 4 shows plots of Mean Arterial Pressure from baseline vs. time for Test Article 1 in Table 1 vs. saline at 5 ml/kg (t=baseline is the time where an initial MAP of the animal is taken; t=0 is the time where bleeding is induced; t=10 is the time where an intravenous test sample is infused).

FIG. 5 shows plots of Mean Arterial Pressure from baseline vs. time for various resuscitative mixtures (t=baseline is the time where an initial MAP of the animal is taken; t=0 is the time where bleeding is induced; t=10 is the time where an intravenous test sample is infused).

FIG. 6 shows plots of Mean Arterial Pressure from baseline vs. time for an exemplary resuscitative mixture (Test Article 1 in Table 1) vs. saline at 1.25 ml/kg (t=baseline is the time where an initial MAP of the animal is taken; t=0 is the time where bleeding is induced; t=10 is the time where an intravenous test sample is infused).

FIG. 7 shows plots of Mean Arterial Pressure from baseline vs. time for various concentrations of an exemplary resuscitative mixture (Test Article 1 in Table 1) (t=baseline is the time where an initial MAP of the animal is taken; t=0 is the time where bleeding is induced; t=10 is the time where an intravenous test sample is infused).

### DETAILED DESCRIPTION

Reference will now be made in detail to various exemplary embodiments provided herein. It is to be understood that the following discussion of exemplary embodiments is not intended as a limitation on the invention, as broadly disclosed herein. Rather, the following discussion is provided to give the reader a more detailed understanding of certain aspects and features of the invention.

Before specific embodiments are described in detail, it is to be understood that the terminology used herein is for the purpose of describing particular embodiments only, and is not intended to be limiting. Further, where a range of values is provided, it is understood that each intervening value between the upper and lower limits of that range is also specifically disclosed. Each smaller range between any stated value or intervening value in a stated range and any other stated or intervening value in that stated range is encompassed and implicitly disclose within this document.

Unless defined otherwise, all technical and scientific terms used herein have the same meaning as commonly understood by one of ordinary skill in the art to which the term belongs. Although any methods and materials similar or equivalent to those described herein can be used in the practice or testing of the various embodiments described herein, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a colloid” includes a plurality of such colloids and reference to “a saccharide” includes reference to one or more saccharides and equivalents thereof known to those skilled in the art. Solely for the sake of additional clarity, at times the terms “at least one” and “one or more” are used to indicate that one or more of the substances referred to can be present. Use of such terminology does not alter or contradict the statements made above in this paragraph. Furthermore, the use of terms that can be described using equivalent terms include the use of those equivalent terms.

In some embodiments, provided herein are compositions comprising one or more of platelets (or platelet-derived material), plasma, or a mixture comprising a buffer, at least one saccharide, at least one salt and at least one high molecular weight, non-ionic, hydrophilic polymer, as well as methods for treating hypovolemia with such compositions. In some embodiments, provided herein are compositions comprising two or more of platelets (or platelet-derived material), plasma, or a mixture comprising a buffer, at least one saccharide, at least one salt and at least one high molecular weight, non-ionic, hydrophilic polymer, as well as methods for treating hypovolemia with such compositions. In some embodiments, provided herein are compositions comprising platelets, plasma, and a mixture comprising a buffer, at least one saccharide, at least one salt and at least one high molecular weight, non-ionic, hydrophilic polymer, as well as methods for treating hypovolemia with such compositions.

In some embodiments, provided herein is a method of treating hypovolemia, comprising administering to a subject a composition as disclosed herein.

In some embodiments, provided herein is a composition of matter comprising: dried blood plasma substantially free of blood cell components; and
a) a resuscitative mixture comprising at least one saccharide, at least one salt and at least one high molecular weight, non-ionic, hydrophilic polymer,
b) platelets or platelet-derived material,

or
c) both a) and b).

In some embodiments, a composition of matter as disclosed herein further comprises an aqueous liquid. In some embodiments, the aqueous liquid is water or saline.

In some embodiments, a resuscitative mixture as disclosed herein further comprises an aqueous liquid. In some embodiments, the aqueous liquid is water or saline.

In some embodiments of the compositions of matter as disclosed herein, the dried platelets, platelet-derived material, or both, further contain an aqueous liquid. In some embodiments, the dried platelets, platelet-derived material, or both, are freeze-dried, spray-dried, or freeze spray-dried.

In some embodiments, a composition of matter as disclosed herein does not comprise trehalose.

In some embodiments of the compositions of matter as disclosed herein, the dried blood plasma, when rehydrated with an aqueous liquid, has coagulation factor levels showing all individual factors at 40 international units (IU) or greater.

In some embodiments, the dried blood plasma, when rehydrated with an aqueous liquid, has coagulation factor that include only Factors VII, VIII and IX.

In some embodiments, the dried blood plasma, when rehydrated with an aqueous liquid, has coagulation factor levels showing individual factors VII, VIII and IX associated with blood clotting at 40 international units (IU) or greater.

In some embodiments, provided herein is a composition of matter comprising: blood plasma rehydrated with an aqueous liquid and substantially free of blood cell components; and

a) a resuscitative mixture comprising at least one saccharide, at least one salt and at least one high molecular weight, non-ionic, hydrophilic polymer,

b) platelets or platelet-derived material,

or

c) both a) and b);

wherein the composition of matter comprises sufficient blood plasma, saccharide(s), salt(s), and high molecular weight, non-ionic, hydrophilic polymer(s) to provide a resuscitative effect when administered to a subject in need of blood resuscitation.

In some embodiments of the composition of matter comprising blood plasma rehydrated with an aqueous liquid, the blood plasma has coagulation factor levels showing all individual factors at 40 international units (IU) or greater.

In some embodiments wherein a dried composition is rehydrated, the composition is rehydrated to about the same volume of the composition prior to drying. In some embodiments wherein a dried composition is rehydrated, the composition is rehydrated to about 0.5 times, or about 0.6 times, or about 0.7 times, or about 0.8 times, or about 0.9 times, or about 1.0 times, or about 1.1 times, or about 1.2 times, or about 1.3 times, or about 1.4 times, or about 1.5 times, or about 1.6 times, or about 1.7 times, or about 1.8 times, or about 1.9 times, or about 2.0 times, the volume of the composition prior to drying.

In some embodiments wherein a dried component (such as plasma, platelets, platelet-derived materials, or a mixture comprising at least one salt and at least one high molecular weight, non-ionic, hydrophilic polymer as disclosed herein) of the composition is rehydrated, the component is rehydrated to the same volume of the composition prior to drying. In some some embodiments wherein a component of the composition is rehydrated, the component is rehydrated to about 0.5 times, or about 0.6 times, or about 0.7 times, or about 0.8 times, or about 0.9 times, or about 1.0 times, or about 1.1 times, or about 1.2 times, or about 1.3 times, or about 1.4 times, or about 1.5 times, or about 1.6 times, or about 1.7 times, or about 1.8 times, or about 1.9 times, or about 2.0 times, the volume of the component prior to drying.

In some embodiments of the compositions of matter disclosed herein, the at least one high molecular weight, non-ionic, hydrophilic polymer of the resuscitative mixture is a copolymer of sucrose and epichlorohydrin. In some more particular embodiments, the copolymer is polysucrose.

In some embodiments, a composition of matter as disclosed herein further comprises an additive for preservation of coagulation function. In some embodiments, the additive for preservation of coagulation function L-Carnitine, Propionyl L-Carnitine, Taurine, Glycerophosphocholine (GCP) or Trehalose. In some embodiments, the additive for preservation of coagulation function comprises taurine.

In some embodiments, provided herein is a method of manufacturing dried blood plasma substantially free of blood cell components, comprising:
a) Mixing a plurality of plasma samples to form a first mixture;

and
b) lyophilizing or spray-drying the first mixture to form the dried blood plasma substantially free of blood cell components.

In some embodiments, the method of manufacturing further comprises rehydrating the dried blood plasma with an aqueous liquid, to form a hydrated blood plasma.

In some embodiments, provided herein are compositions comprising one or more of platelets (or platelet-derived material), plasma, or a mixture comprising a buffer, at least one saccharide, at least one salt and at least one high molecular weight, non-ionic, hydrophilic polymer, as well as methods for treating hypovolemia with such compositions. In some embodiments, provided herein are compositions comprising two or more of platelets (or platelet-derived material), plasma, or a mixture comprising a buffer, at least one saccharide, at least one salt and at least one high molecular weight, non-ionic, hydrophilic polymer, as well as methods for treating hypovolemia with such compositions. In some embodiments, provided herein are compositions comprising platelets (or platelet-derived material), plasma, and a mixture comprising a buffer, at least one saccharide, at least one salt and at least one high molecular weight, non-ionic, hydrophilic polymer, as well as methods for treating hypovolemia with such compositions.

Any one of the compositions provided herein can comprise red blood cells or red blood cell substitutes. In some embodiments, provided herein is a composition comprising one or more of platelets or platelet-derived material, plasma, or a mixture comprising at least one salt and at least one high molecular weight, non-ionic, hydrophilic polymer, and further comprising red blood cells or red blood cell substitutes. Thus, in some embodiments, provided herein is a composition of matter comprising:

red blood cells or red blood cell substitutes; and

a) a resuscitative mixture comprising at least one salt and at least one high molecular weight, non-ionic, hydrophilic polymer,
b) platelets or platelet-derived material,

or
c) both a) and b).

In some embodiments, provided herein is a composition of matter comprising dried blood plasma substantially free of blood cell components, red blood cells or red blood cell substitutes;

and
a) a resuscitative mixture comprising at least one salt and at least one high molecular weight, non-ionic, hydrophilic polymer,
b) platelets or platelet-derived material,

or
c) both a) and b).

In some embodiments, provided herein is a composition of matter comprising:

red blood cells or red blood cell substitutes; and
a) a resuscitative mixture comprising at least one salt and at least one high molecular weight, non-ionic, hydrophilic polymer,

and
b) platelets or platelet-derived material,

In some embodiments, provided herein is a composition of matter comprising:

red blood cells or red blood cell substitutes; and
a resuscitative mixture comprising at least one salt and at least one high molecular weight, non-ionic, hydrophilic polymer.

In some embodiments, provided herein is a composition of matter comprising: dried blood plasma substantially free of blood cell components;

red blood cells or red blood cell substitutes; and
a) a resuscitative mixture comprising at least one salt and at least one high molecular weight, non-ionic, hydrophilic polymer,

and
b) platelets or platelet-derived material,

In some embodiments, provided herein is a composition of matter comprising:

dried blood plasma substantially free of blood cell components;

red blood cells or red blood cell substitutes; and
a resuscitative mixture comprising at least one salt and at least one high molecular weight, non-ionic, hydrophilic polymer.

In some embodiments, provided herein is a composition of matter comprising:

dried blood plasma substantially free of blood cell components;
a) a resuscitative mixture comprising at least one salt and at least one high molecular weight, non-ionic, hydrophilic polymer,

and
b) platelets or platelet-derived material,

In some embodiments, provided herein is a composition of matter comprising:

dried blood plasma substantially free of blood cell components; and
a resuscitative mixture comprising at least one salt and at least one high molecular weight, non-ionic, hydrophilic polymer.

In some embodiments, provided herein is a method of treating hypovolemia, comprising administering to a subject a composition as disclosed herein.

In some embodiments, provided herein is a composition comprising platelets or platelet-derived material. In some embodiments, provided herein is a method of treating hypovolemia in a subject, comprising administering to the subject a composition comprising platelets or platelet-derived material. In some embodiments, the platelets or platelet-derived material are dried. In some embodiments, the platelets or platelet-derived material are freeze-dried. In some embodiments, the platelets or platelet-derived material are not freeze-dried. In some embodiments, the composition comprising the platelets are aqueous. In some embodiments, the platelet-derived material is dried. In some embodiments, the platelet-derived material is freeze-dried. In some embodiments, the composition comprising the platelet-derived material is aqueous.

In some embodiments, provided herein is a composition comprising plasma. In some embodiments, provided herein is a method of treating hypovolemia in a subject, comprising administering to the subject a composition comprising plasma. In some embodiments, the plasma is dried. In some embodiments, the plasma is freeze-dried. In some embodiments, the plasma is not freeze-dried. In some embodiments, the composition comprising the plasma is aqueous. In some embodiments, the plasma is dried blood plasma substantially free of blood cell components.

In some embodiments, provided herein is a composition comprising a mixture comprising at least one salt and at least one high molecular weight, non-ionic, hydrophilic polymer. In some embodiments, provided herein is a composition comprising a mixture comprising a buffer, at least one saccharide, at least one salt and at least one high molecular weight, non-ionic, hydrophilic polymer. In some embodiments, provided herein is a method of treating hypovolemia in a subject, comprising administering to the subject a composition comprising a mixture comprising at least one salt and at least one high molecular weight, non-ionic, hydrophilic polymer. In some embodiments, provided herein is a method of treating hypovolemia in a subject, comprising administering to the subject a composition comprising a mixture comprising a buffer, at least one saccharide, at least one salt, and at least one high molecular weight, non-ionic, hydrophilic polymer. In some embodiments, the mixture is dried. In some embodiments, the mixture is freeze-dried. In some embodiments, the mixture is not freeze-dried. In some embodiments, the mixture is aqueous. In some embodiments, the mixture comprising a buffer, at least one saccharide, at least one salt, and at least one high molecular weight, non-ionic, hydrophilic polymer is Reovol™.

In some embodiments, provided herein is a composition comprising (1) platelets or platelet-derived material, and (2) plasma. In some embodiments, provided herein is a method of treating hypovolemia in a subject, comprising administering to the subject a composition comprising (1) platelets or platelet-derived material, and (2) plasma. In some embodiments, the platelets are dried (e.g., freeze-dried). In some embodiments, the platelet-derived material is dried. In some embodiments, the plasma is dried (e.g., freeze-dried). In some embodiments, the platelets are dried and the plasma is dried. In some embodiments, the platelet-derived material is dried and the plasma is dried. In some embodiments, the composition comprising (1) platelets or platelet-derived material, and (2) plasma, is aqueous. In some embodiments, the composition comprises platelets and plasma, wherein the plasma is dried blood plasma substantially free of blood cell components. In some embodiments, the composition comprises platelet-derived material and plasma, wherein the plasma is dried blood plasma substantially free of blood cell components. In some embodiments, the composition comprising (1) platelets or platelet-derived material, and (2) plasma, does not comprise a mixture comprising at least one salt and at least one high molecular weight, non-ionic, hydrophilic polymer. In some embodiments, the composition comprising (1) platelets or platelet-derived material, and (2) plasma, does not comprise a mixture, wherein the mixture comprises a buffer, at least one saccharide, at least one salt, and at least one high molecular weight, non-ionic, hydrophilic polymer.

In some embodiments, provided herein is a composition comprising (1) platelets or platelet-derived material, and (2) a mixture comprising at least one salt and at least one high molecular weight, non-ionic, hydrophilic polymer. In some embodiments, provided herein is a composition comprising (1) platelets or platelet-derived material, and (2) a mixture comprising a buffer, at least one saccharide, at least one salt, and at least one high molecular weight, non-ionic, hydrophilic polymer. In some embodiments, provided herein is a method of treating hypovolemia in a subject, comprising administering to the subject a composition comprising (1) platelets or platelet-derived material, and (2) a mixture comprising at least one salt, and at least one high molecular weight, non-ionic, hydrophilic polymer. In some embodiments, provided herein is a method of treating hypovolemia in a subject, comprising administering to the subject a composition comprising (1) platelets or platelet-derived material, and (2) a mixture comprising a buffer, at least one saccharide, at least one salt, and at least one high molecular weight, non-ionic, hydrophilic polymer. In some embodiments, the platelets are dried (e.g., freeze-dried). In some embodiments, the platelet-derived material is dried (e.g., freeze-dried). In some embodiments, the mixture is dried (e.g., freeze-dried). In some embodiments, the platelets are dried and the mixture is dried. In some embodiments, the platelet-derived material is dried and the mixture is dried. In some embodiments, the composition comprising (1) platelets or platelet-derived material, and (2) a mixture comprising at least one salt, and at least one high molecular weight, non-ionic, hydrophilic polymer is aqueous. In some embodiments, the composition comprising (1) platelets or platelet-derived material, and (2) a mixture comprising a buffer, at least one saccharide, at least one salt, and at least one high molecular weight, non-ionic, hydrophilic polymer is aqueous. In some embodiments, the composition comprises platelets and the mixture comprising a buffer, at least one saccharide, at least one salt, and at least one high molecular weight, non-ionic, hydrophilic polymer is Reovol™. In some embodiments, the compositions comprises platelet-derived material and the mixture comprising a buffer, at least one saccharide, at least one salt, and at least one high molecular weight, non-ionic, hydrophilic polymer is Reovol™. In some embodiments, the composition comprising (1) platelets or platelet-derived material, and (2) a mixture comprising a buffer, at least one saccharide, at least one salt, and at least one high molecular weight, non-ionic, hydrophilic polymer, does not comprise plasma.

In some embodiments, provided herein is a composition comprising (1) plasma and (2) a mixture comprising at least one salt and at least one high molecular weight, non-ionic, hydrophilic polymer. In some embodiments, provided herein is a composition comprising (1) plasma and (2) a mixture comprising a buffer, at least one saccharide, at least one salt, and at least one high molecular weight, non-ionic, hydrophilic polymer. In some embodiments, provided herein is a method of treating hypovolemia in a subject, comprising administering to the subject a composition comprising (1) plasma and (2) a mixture comprising at least one salt and at least one high molecular weight, non-ionic, hydrophilic polymer. In some embodiments, provided herein is a method of treating hypovolemia in a subject, comprising administering to the subject a composition comprising (1) plasma and (2) a mixture comprising a buffer, at least one saccharide, at least one salt, and at least one high molecular weight, non-ionic, hydrophilic polymer. In some embodiments, the plasma is dried (e.g., freeze-dried). In some embodiments, the mixture comprising at least one salt and at least one high molecular weight, non-ionic, hydrophilic polymer is dried (e.g., freeze-dried). In some embodiments, the mixture comprising a buffer, at least one saccharide, at least one salt, and at least one high molecular weight, non-ionic, hydrophilic polymer is dried (e.g., freeze-dried). In some embodiments, the plasma is dried and the mixture comprising a buffer, at least one saccharide, at least one salt, and at least one high molecular weight, non-ionic, hydrophilic polymer is dried. In some embodiments, the plasma is dried and the mixture comprising a buffer, at least one saccharide, at least one salt, and at least one high molecular weight, non-ionic, hydrophilic polymer is dried. In some embodiments, the composition comprising (1) plasma and (2) a mixture comprising at least one salt and at least one high molecular weight, non-ionic, hydrophilic polymer is aqueous. In some embodiments, the composition comprising (1) plasma and (2) a mixture comprising a buffer, at least one saccharide, at least one salt, and at least one high molecular weight, non-ionic, hydrophilic polymer is aqueous. In some embodiments, the plasma is dried blood plasma substantially free of blood cell components, and the mixture comprising a buffer, at least one saccharide, at least one salt, and at least one high molecular weight, non-ionic, hydrophilic polymer is Reovol™. In some embodiments, the composition comprising (1) plasma and (2) a mixture comprising at least one salt and at least one high molecular weight, non-ionic, hydrophilic polymer, does not comprise platelets or platelet-derived material. In some embodiments, the composition comprising (1) plasma and (2) a mixture comprising a buffer, at least one saccharide, at least one salt, and at least one high molecular weight, non-ionic, hydrophilic polymer, does not comprise platelets or platelet-derived material.

In some embodiments of compositions comprising two or more of platelets (or platelet-derived material), plasma, or a mixture comprising at least one salt and at least one high molecular weight, non-ionic, hydrophilic polymer, the plasma is present in an amount from about 1% to about 99% wt/v, about 10% to about 90% wt/v, such as from about 20% to about 80% wt/v, such as from about 30% to about 80% wt/v, such as from about 30% to about 70% wt/v, such as from about 30% to about 60% wt/v, such as from about 40% to about 60% wt/v, such as from about 40% to about 50% wt/v; the platelets (or platelet-derived material) are present in a concentration of about 10,000 platelets/μL to about 2,000,000 platelets/μL, such as from about 20,000 platelets/μL to about 1,500,000 platelets/μL, such as from about 30,000 platelets/μL to about 1,000,000 platelets/μL, such as from about 40,000 platelets/μL to about 900,000 platelets/μL v, such as from about 50,000 platelets/μL to about 800,000 platelets/μL, such as from about 100,000 platelets/μL to about 700,000 platelets/μL, such as from about 200,000 platelets/μL to about 600,000 platelets/μL, such as from about 300,000 platelets/μL to about 500,000 platelets/μL; and the mixture comprising at least one salt and at least one high molecular weight, non-ionic, hydrophilic polymer is present in an amount from about 0% to about 90% wt/v, such as from about 10% to about 80% wt/v, such as from about 20% to about 80% wt/v, such as from about 30% to about 80% wt/v, such as from about 30% to about 70% wt/v, such as from about 30% to about 60% wt/v, such as from about 40% to about 60% wt/v, such as from about 40% to about 50% wt/v;

wherein wt/v indicates the ratio between the weight of the component and the weight of the composition;

provided that at least one of:
platelets (or platelet-derived material), or
a mixture comprising at least one salt and at least one high molecular weight, non-ionic, hydrophilic polymer, is present in the composition.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 30% to about 80% wt/v; and
the platelets (or platelet-derived material) are present in a concentration of about 10,000 platelets/μL to about 2,000,000 platelets/μL.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 40% to about 70% wt/v; and
the platelets (or platelet-derived material) are present in a concentration of about 100,000 platelets/μL to about 1,000,000 platelets/μL.

In some embodiments of compositions comprising two or more of platelets (or platelet-derived material), plasma, or a mixture comprising at least one salt and at least one high molecular weight, non-ionic, hydrophilic polymer,

the plasma is present in an amount from about 30% to about 80% wt/v; and
the mixture comprising at least one salt and at least one high molecular weight, non-ionic, hydrophilic polymer, is present in an amount from about 20% to about 70% wt/v

In some embodiments of compositions comprising two or more of platelets (or platelet-derived material), plasma, or a mixture comprising at least one salt and at least one high molecular weight, non-ionic, hydrophilic polymer,

the plasma is present in an amount from about 40% to about 70% wt/v; and the mixture comprising at least one salt and at least one high molecular weight, non-ionic, hydrophilic polymer, is present in an amount from about 30% to about 60% wt/v.

In some embodiments of compositions comprising two or more of platelets (or platelet-derived material), plasma, or a mixture comprising at least one salt and at least one high molecular weight, non-ionic, hydrophilic polymer,

the plasma is dried;
and
the mixture comprising at least one salt and at least one high molecular weight, non-ionic, hydrophilic polymer, is in liquid form.

In some embodiments of compositions comprising two or more of platelets (or platelet-derived material), plasma, or a mixture comprising at least one salt and at least one high molecular weight, non-ionic, hydrophilic polymer,

the platelets (or platelet-derived material) are dried;
and
the mixture comprising at least one salt and at least one high molecular weight, non-ionic, hydrophilic polymer, is in liquid form.

In some embodiments, provided herein is a composition comprising (1) platelets or platelet-derived material, (2) plasma, and (3) a mixture comprising at least one salt and at least one high molecular weight, non-ionic, hydrophilic polymer. In some embodiments, provided herein is a composition comprising (1) platelets or platelet-derived material, (2) plasma, and (3) a mixture comprising a buffer, at least one saccharide, at least one salt, and at least one high molecular weight, non-ionic, hydrophilic polymer. In some embodiments, provided herein is a method of treating hypovolemia in a subject, comprising administering to the subject a composition comprising (1) platelets or platelet-derived material, (2) plasma, and (3) a mixture comprising at least one salt and at least one high molecular weight, non-ionic, hydrophilic polymer. In some embodiments, provided herein is a method of treating hypovolemia in a subject, comprising administering to the subject a composition comprising (1) platelets or platelet-derived material, (2) plasma, and (3) a mixture comprising a buffer, at least one saccharide, at least one salt, and at least one high molecular weight, non-ionic, hydrophilic polymer. In some embodiments, the platelets are dried (e.g., freeze-dried). In some embodiments, the platelet-derived material is dried (e.g., freeze-dried). In some embodiments, the plasma is dried (e.g., freeze-dried). In some embodiments, the mixture comprising at least one salt and at least one high molecular weight, non-ionic, hydrophilic polymer is dried (e.g., freeze-dried). In some embodiments, the mixture comprising a buffer, at least one saccharide, at least one salt, and at least one high molecular weight, non-ionic, hydrophilic polymer is dried (e.g., freeze-dried). In some embodiments, the platelets are dried, the plasma is dried, and the mixture comprising at least one salt and at least one high molecular weight, non-ionic, hydrophilic polymer is dried. In some embodiments, the platelets are dried, the plasma is dried, and the mixture comprising a buffer, at least one saccharide, at least one salt, and at least one high molecular weight, non-ionic, hydrophilic polymer is dried. In some embodiments, the platelet-derived material is dried, the plasma is dried, and the mixture comprising at least one salt and at least one high molecular weight, non-ionic, hydrophilic polymer is dried. In some embodiments, the platelet-derived material is dried, the plasma is dried, and the mixture comprising a buffer, at least one saccharide, at least one salt, and at least one high molecular weight, non-ionic, hydrophilic polymer is dried. In some embodiments, the composition comprising (1) platelets or platelet-derived material, (2) plasma, and (3) the mixture comprising at least one salt and at least one high molecular weight, non-ionic, hydrophilic polymer, is aqueous. In some embodiments, the composition comprising (1) platelets or platelet-derived material, (2) plasma, and (3) the mixture comprising a buffer, at least one saccharide, at least one salt, and at least one high molecular weight, non-ionic, hydrophilic polymer, is aqueous. In some embodiments, the plasma is dried blood plasma substantially free of blood cell components, and the mixture comprising a buffer, at least one saccharide, at least one salt, and at least one high molecular weight, non-ionic, hydrophilic polymer is Reovol™. In some embodiments, the plasma is dried blood plasma substantially free of blood cell components, and the mixture comprising a buffer, at least one saccharide, at least one salt, and at least one high molecular weight, non-ionic, hydrophilic polymer is Reovol™.

In some embodiments of compositions comprising platelets (or platelet-derived material), plasma, and a mixture comprising at least one salt and at least one high molecular weight, non-ionic, hydrophilic polymer, the plasma is present in an amount from about 1% to about 99% wt/v, 10% to about 90% wt/v, such as from about 20% to about 80% wt/v, such as from about 30% to about 80% wt/v, such as from about 30% to about 70% wt/v, such as from about 30% to about 60% wt/v, such as from about 40% to about 60% wt/v, such as from about 40% to about 50% wt/v; the platelets (or platelet-derived material) are present in a concentration of about 10,000 platelets/μL to about 2,000,000 platelets/μL, such as from about 20,000 platelets/μL to about 1,500,000 platelets/μL, such as from about 30,000 platelets/μL to about 1,000,000 platelets/μL, such as from about 40,000 platelets/μL to about 900,000 platelets/μL v, such as from about 50,000 platelets/μL to about 800,000 platelets/μL, such as from about 100,000 platelets/μL to about 700,000 platelets/μL, such as from about 200,000 platelets/μL to about 600,000 platelets/μL, such as from about 300,000 platelets/μL to about 500,000 platelets/μL; and the mixture comprising at least one salt and at least one high molecular weight, non-ionic, hydrophilic polymer is present in an amount from about 1% to about 99% wt/v, such as from about 10% to about 90% wt/v, such as from about 20% to about 80% wt/v, such as from about 30% to about 80% wt/v, such as from about 30% to about 70% wt/v, such as from about 30% to about 60% wt/v, such as from about 40% to about 60% wt/v, such as from about 40% to about 50% wt/v;

wherein wt/v indicates the ratio between the weight of the component and the weight of the composition.

In some embodiments of compositions comprising platelets (or platelet-derived material), plasma, and a mixture comprising at least one salt and at least one high molecular weight, non-ionic, hydrophilic polymer,

the plasma is present in an amount from about 40% to about 60% wt/v; and the platelets (or platelet-derived material) are present in a concentration of about 10,000 platelets/μL to about 800,000 platelets/μL; and
the mixture comprising at least one salt and at least one high molecular weight, non-ionic, hydrophilic polymer, is present in an amount from about 20% to about 40% wt/v.

In some embodiments of compositions comprising platelets (or platelet-derived material), plasma, and a mixture comprising at least one salt and at least one high molecular weight, non-ionic, hydrophilic polymer,

the plasma is present in an amount from about 30% to about 50% wt/v; and
the platelets (or platelet-derived material) are present in a concentration of about 100,000 platelets/μL to about 800,000 platelets/μL; and
the mixture comprising at least one salt and at least one high molecular weight, non-ionic, hydrophilic polymer, is present in an amount from about 30% to about 40% wt/v.

In some embodiments of compositions comprising platelets (or platelet-derived material), plasma, and a mixture comprising at least one salt and at least one high molecular weight, non-ionic, hydrophilic polymer,

the plasma is dried;
the platelets (or platelet-derived material) are dried;
and
the mixture comprising at least one salt and at least one high molecular weight, non-ionic, hydrophilic polymer, is in liquid form.

In some embodiments of the composition comprising one or more of platelets (or platelet-derived material), plasma, or a mixture comprising a buffer, at least one saccharide, at least one salt, and at least one high molecular weight, non-ionic, hydrophilic polymer; of the composition comprising two or more of platelets (or platelet-derived material), plasma, or a mixture comprising a buffer, at least one saccharide, at least one salt, and at least one high molecular weight, non-ionic, hydrophilic polymer, as well as methods for treating hypovolemia with such compositions; or of the composition comprising platelets (or platelet-derived material), plasma, and a mixture comprising a buffer, at least one saccharide, at least one salt, and at least one high molecular weight, non-ionic, hydrophilic polymer, the composition is in infusible or injectable form.

Accordingly, in some embodiments, provided herein is a method for treating hypovolemia with an infusible form or an injectable form of the composition as disclosed herein. In some embodiments, the mixture comprising at least one salt and at least one high molecular weight, non-ionic, hydrophilic polymer comprises ethanol. In some embodiments, the mixture comprising at least one salt and at least one high molecular weight, non-ionic, hydrophilic polymer does not comprise ethanol and does not comprise trehalose.

In some embodiments of the mixture comprising at least one salt and at least one high molecular weight, non-ionic, hydrophilic polymer, the at least one salt is a sodium salt.

In some embodiments, the mixture comprising a buffer, at least one saccharide, at least one salt, and at least one high molecular weight, non-ionic, hydrophilic polymer comprises ethanol. In some embodiments, the mixture comprising at least one salt and at least one high molecular weight, non-ionic, hydrophilic polymer does not comprise ethanol.

In some embodiments of the mixture comprising a buffer, at least one saccharide, at least one salt, and at least one high molecular weight, non-ionic, hydrophilic polymer, the at least one salt is a sodium salt.

In some embodiments of the mixture comprising a buffer, at least one saccharide, at least one salt, and at least one high molecular weight, non-ionic, hydrophilic polymer, the saccharide is trehalose.

In some embodiments, the mixture provided herein includes one or more salts, such as phosphate salts, sodium salts (e.g., NaCl), potassium salts (e.g., KCl), calcium salts (e.g., CaCl₂)), magnesium salts, and any other salt that can be found in blood or blood products, or that is known to be useful in cryopreserving platelets, or any combination of two or more of these. In some embodiments, the one or more salts are present in an amount of about 0.01% (wt/v) to about 10% (wt/v) (e.g., such as about 0.02% (wt/v) to about 8% (wt/v), about 0.03% (wt/v) to about 5% (wt/v), about 0.4% (wt/v) to about 2% (wt/v), or about 0.5% (wt/v) to about 1% (wt/v)) of the composition. In some embodiments, the one or more salts are present in an amount of about 0.1% (wt/v) to about 5% (wt/v) of the composition. In some embodiments, the one or more salts are present in an amount of about 0.1% (wt/v) or less. In some embodiments an effective amount of the one or more salts is an amount of salts as disclosed herein above.

In some embodiments, the one or more salts are present in an amount that adjusts and/or maintains a composition at an osmolality level that is equivalent to blood osmolality, or plasma osmolality. For example, in some embodiments, the one or more salts are present in an amount that yields a composition at an osmolality level of about 150 mOsm/kg to about 600 mOsm/kg (e.g., such as about 250 to about 300 mOsm/kg). In some embodiments an effective amount of the one or more salts is an amount of salts that yields a composition at an osmolality level as disclosed herein above.

In some embodiments, the mixture provided herein includes one or more buffer materials. In some embodiments, the buffer is selected from the group consisting of phosphate buffered saline (PBS), bicarbonate/carbonic acid, such as a sodium-bicarbonate buffer, N-2-hydroxyethylpiperazine-N′-2-ethanesulfonic acid (HEPES), a tris-based buffer, a tris-buffered saline (TB S), and combinations thereof. In some embodiments, the one or more buffer materials are present in an amount of about 0.01% (wt/v) to about 30% (wt/v) (e.g., such as about 0.02% (wt/v) to about 20% (wt/v), about 0.03% (wt/v) to about 10% (wt/v), about 0.4% (wt/v) to about 5% (wt/v), or about 0.5% (wt/v) to about 2% (wt/v)) of the composition. In some embodiments, the one or more buffer materials are present in an amount of about 0.5% (wt/v) to about 2% (wt/v) of the composition. In some embodiments, the one or more buffer materials are present in an amount of about 0.1% (wt/v) or less. In some embodiments an effective amount of the one or more buffer materials is an amount of one or more buffer materials as disclosed herein above.

In some embodiments, the mixture provided herein includes an organic solvent. In some embodiments, the organic solvent is present in an amount of about 0.1% (wt/v) to about 5% (wt/v) of the composition. In some embodiments, the organic solvent is present in an amount of about 0.1% (wt/v) to about 1% (wt/v) of the composition. In some embodiments, the organic solvent comprises an alcohol. In some embodiments, the alcohol comprises a short-chain alcohol. In some embodiments, the short-chain alcohol is selected from the group consisting of methanol, ethanol, propanol, 1-propanol, 2-propanol, and combinations thereof.

In some embodiments, the mixture provided herein includes a component selected from the group consisting of a saccharide, a monosaccharide, a disaccharide, and combinations thereof. In some embodiments, the mixture comprises a component selected from the group consisting of sucrose, maltose, trehalose, glucose, mannose, xylose, dextrose, and combinations thereof. In some embodiments, the one or more components are present in an amount of about 0.01% (wt/v) to about 60% (wt/v) of the composition. In some embodiments an effective amount of the component selected from the group consisting of a saccharide, a monosaccharide, a disaccharide, and combinations thereof is an amount of one or more components as disclosed herein above. In some embodiments, the one or more buffer materials are present in an amount of about 0.1% (wt/v) to about 40% (wt/v), about 30% (wt/v) to about 40% (wt/v), or about 0.02% (wt/v) to about 1% (wt/v), of the composition.

In some embodiments, the mixture provided herein includes a polymer derived from sucrose and epichlorohydrin. In some embodiments, the polymer is a polysucrose. In some embodiments, the polysucrose is in an amount of about 1% (w/v) to about 30% (w/v) of the mixture, or 1% (v/v) to about 30% (v/v) of the mixture. In some embodiments, the polysucrose is in an amount of about 3% (w/v) to about 20% (w/v), or about 3% (v/v) to about 20% (v/v). In some embodiments, the polysucrose is in an amount of about 5% (w/v) to about 10% (w/v), or about 5% (v/v) to about 10% (v/v). In some embodiments an effective amount of the polymer derived from sucrose and epichlorohydrin is an amount as disclosed herein above. In some embodiments, the polysucrose comprises a high molecular weight polymer. In some embodiments, the high molecular weight polymer has an average molecular weight of at least about 70 kilodaltons (kDa), of at least about 400 kilodaltons, of at least about 600 kilodaltons, of at least about 800 kilodaltons, of at least about 1,000 kilodaltons, or of at least about 2,000 kilodaltons. In some embodiments, the polymers are made by the copolymerization of sucrose and epichlorohydrin, such as a polysucrose. In some embodiments, the polymer is polysucrose with an average molecular weight from about 70 kD to about 2 megadaltons (MD), such as from about 200 kD to about 1 MD, such as from about 70 kD to about 400 kD, such as from about 200 kD to about 400 kD.

In some embodiments, a mixture comprising a buffer, at least one saccharide, at least one salt, and at least one high molecular weight, non-ionic, hydrophilic polymer is one of the four mixtures (“Test Article”) shown in Table 1 below:

TABLE 1

| | | | Test Article | | | |
|---|---|---|---|---|---|---|
| | | | 1 | 2 | 3 | 4 |
| Component | Salt | NaCl | 3.5 | 3.5 | 3.5 | 7.4 |
| (mg/mL) | | KCl | 0.3 | 0.3 | 0.3 | 0.3 |
| | Buffer | HEPES | 1.8 | 1.8 | 1.8 | 1.8 |
| | | Sod Bicarb | 0.8 | 0.8 | 0.8 | 0.8 |
| | Alcohol | Ethanol | 0.3 | 0.3 | 0 | 0 |
| | Sugar | Dextrose | 0.4 | 0.4 | 0.4 | 0.4 |
| | | Trehalose | 30.3 | 30.3 | 0 | 0 |
| | Polymer | Polysucrose | 60 | 60 | 60 | 60 |
| | | w/average | | | | |
| | | molecular weight | | | | |
| | | of 400 kD | | | | |

In some embodiments, the compositions of matter provided herein comprise dried blood plasma in combination with one or more additional components, wherein such additional components may comprise (a) a mixture comprising a buffer, at least one saccharide, at least one salt, and at least one high molecular weight, non-ionic, hydrophilic polymer; (b) platelets or platelet-derived material; or (c) both (a) and (b). In some embodiments, the compositions of matter are very well suited for use in ameliorative or prophylactic in vivo treatment for blood volume deficits.

In exemplary embodiments, an additional component comprises a colloid-based resuscitative composition that provides blood volume expansion when administered to a subject. In some embodiments, the colloid-based resuscitative composition can be the compositions disclosed in PCT/US2016/036657 (published as WO 2016/201081) and/or PCT/US2016/065681, incorporated herein in their entirety, or one derived from those compositions. It is to be understood that other colloid-based resuscitative mixtures are encompassed in some embodiments. In some embodiments, some colloid-based resuscitative mixtures containing hetastarch, such as Hextend® (Biotime, Inc., Alameda, Calif.), are excluded from compositions provided herein. In some embodiments, mixtures containing hetastarch are excluded from compositions provided herein.

In some embodiments, an exemplary additional component to the dried blood plasma is dried platelets, such as freeze-dried platelets, spray-dried platelets, and/or spray freeze-dried platelets, etc, or dried platelet-derived material. Biological materials that are not intact platelets but result from the drying process of platelets are encompassed in some embodiments. Freeze-dried platelets provided herein include those prepared by the methods disclosed in U.S. Pat. Nos. 7,811,558, 8,486,617, and 8,097,403, for example.

In some embodiments, kits containing the components of compositions of matter provided herein. As used in this document, a kit is a package that provides some or all of the components of the composition of matter provided herein. The package is suitable for shipping, delivering, etc. the composition components. The package can also be suitable for storage of the composition components until needed for use. Kits for shipping and storage of blood components are described in more detail below.

### EXAMPLES

The Examples provided herein are intended to be purely exemplary of certain embodiments of the invention, and should not be considered as limiting the invention in any way.

### Example 1

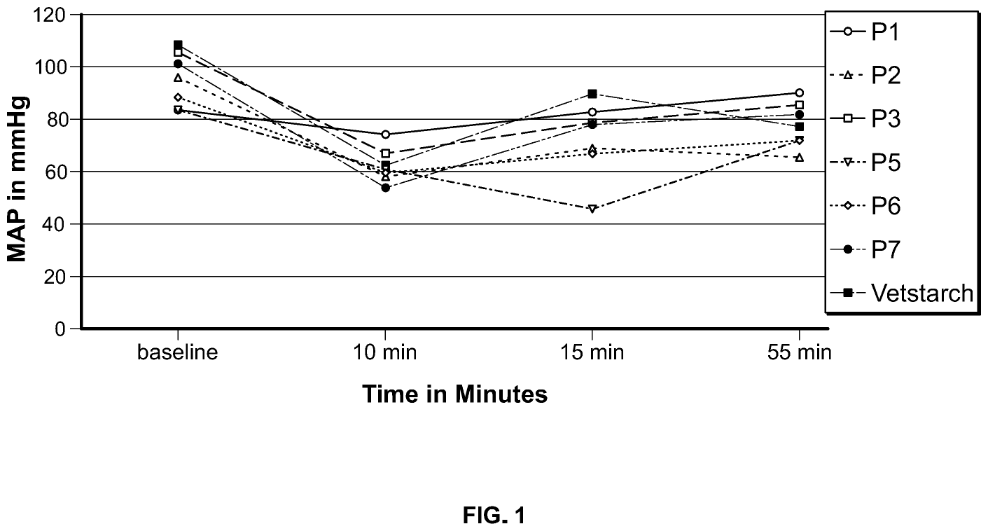

Bleeding was Induced in 4 Subjects (Male Sprague Dawley Rats) at Time t=0 and a Composition as Shown in FIG. 1 was Administered to a Subject.

Referring to FIG. 1, the compositions are as follows:
P1=the mixture of Table 1, col. 1, in solid form
P2=the mixture of Table 1, col. 2, in liquid form
P3=the mixture of Table 1, col. 3, original solid form rehydrated with plasma to the same volume as in P2.
P4=the liquid formulation of P3
P5=platelet derived material (thrombosomes) and the mixture of Table 1, col. 1, and saline solution
P6=plasma and platelet-derived material (thrombosomes), and the mixture of Table 1, col. 1.
P7=platelet-derived material (thrombosomes) and the mixture of Table 1, col. 1, plasma, and the mixture of Table 1, col. 3, originally in solid form, rehydrated with plasma to the same volume as in P2
Vetstarch® (commercially available form of 6% Hydroxyethyl Starch 130/0.4 in 0.9% Sodium Chloride)

The following is an exemplary protocol showing how the rehydrated composition P3 is prepared:

### Step 1: Prepare Mixture and Plasma for Rehydration

1. Place the mixture of Table 1, col. 3 in a 15 mL vial.
2. Filter Rat plasma using a 0.22 μm filter before rehydration)
Step 2: Rehydration of the Mixture (Table 1, col. 3) with Plasma.
Draw 14.3 mL of rat plasma using a sterile syringe and needle. This volume is specific to the 15 ml vial fill. The calculations and dilutions were performed to infuse 1.89×10⁸ platelets per every kg of the subject (i.e., subject weight). Such an infusion is consistent with the standard human dosing amount of 3×10¹¹/70 kg (subject weight) and certain industry standards, for example, as disclosed in Optimal Fluid Therapy for Traumatic Hemorrhagic Shock, Holcomb et. al., Crit Care Clin. 2017; 33(1): 15-36, which is incorporated by reference herein in its entirety.
   2. Rehydrate the mixture contained in the vial with the appropriate volume of rat plasma, directing the stream of water against the glass side of the vial.
   3. Record the start time of rehydration.
   4. Gently swirl the rehydrated mixture for approximately 5-10 seconds to assist in the rehydration process.
   5. Allow the rehydrated mixture to rest for 10 minutes at room temperature to complete the rehydration process before use.
   6. Swirl the rehydrated mixture every few minutes (at approximately 5 minutes) while rehydrating.
   7. If clumps are observed, swirl the rehydrated mixture and let rest for another 5 minutes.
   The following is an exemplary protocol showing how the compositions P5-P7 are prepared:
   Step 1: Rehydration with Sterile water.
1. Draw 9.5 ml of sterile water using a sterile syringe and needle. This volume is specific to a 10 ml vial fill
2. Rehydrate the platelets to 10 ml to give ‘Solution A’

### Step 2: Dilution Part 1

Add 1 ml of Solution A obtained in Step 1 to 9 ml of Plasma, Saline, or composition P3 to give ‘Solution B’

### Step 2: Dilution Part 2

Add 1 ml of Solution B obtained in Step 2, part 1 and 4 ml of Plasma, Saline, or P3 to
obtain ‘Solution C’, to achieve a concentration of 1.89×10⁸ platelets per kg.
   Solution C obtained in Step 2, part 2 is injected in the subject in a volume calculated based on the weight of the rat as per the dosage criteria of 5 ml/Kg.
   The rat plasma used was Sprague-Dawley rat plasma manufactured by Innovation Research Inc. (Novi, Mich., USA).

In the alternative, freeze-dried rat plasma may also be used, such as freeze-dried rat plasma (e.g., product number P2516) manufactured by Sigma-Aldrich (St. Louis, Mo., USA).

The resulting plots of MAP vs. time are shown in FIG. 1.

### Example 2

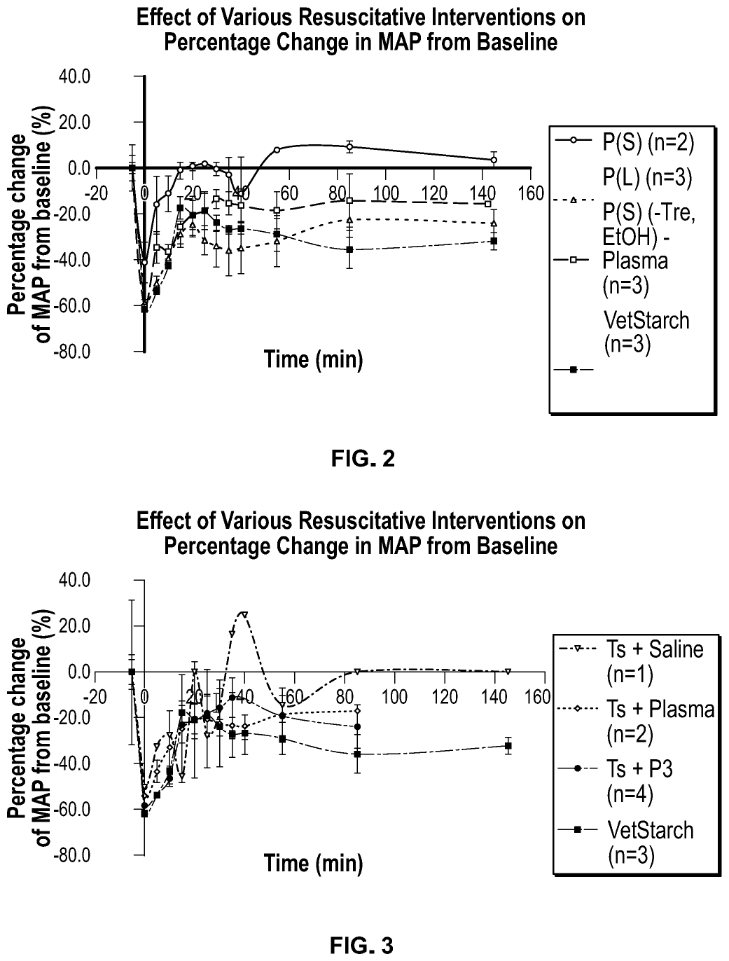

Bleeding was induced in 4 subjects (rats) at time t=0 and a composition as shown in FIG. 2 was administered to a subject at time t=10.
Referring to FIG. 2, the compositions are as follows:
P(S)=the mixture of Table 1, col. 1, in solid form
P(L)=the mixture of Table 1, col. 2, in liquid form
P(S) (−Tre, EtOH)−plasma=the mixture of Table 1, col. 3, original solid form rehydrated with plasma to the same volume as in P2
Vetstarch® (commercially available form of 6% Hydroxyethyl Starch 130/0.4 in 0.9% Sodium Chloride)

The resulting plots of % change of MAP vs. time are shown in FIG. 2.

### Example 3

Bleeding was induced in subjects (rats) at time t=0 and a composition as shown in FIG. 3 was administered to a subject. The number of subjects treated with each composition is shown in the figure.

Referring to FIG. 3, the compositions are as follows:
Ts+saline=platelet derived material (thrombosomes) and the mixture of Table 1, col. 1, and saline solution
Ts+plasma=plasma and platelet-derived material (thrombosomes), and the mixture of Table 1, col. 1.
Ts+P3=platelet-derived material (thrombosomes) and the mixture of Table 1, col. 1, plasma, and the mixture of Table 1, col. 3, originally in solid form, rehydrated with plasma to the same volume as in P2
Vetstarch® (commercially available form of 6% Hydroxyethyl Starch 130/0.4 in 0.9% Sodium Chloride)
The resulting plots of % change of MAP vs. time are shown in FIG. 3.

### Dose Escalation of Combination Resuscitative Mixtures Experiments

Male Sprague Dawley rats weighing approximately 400 gm were used to evaluate dose escalation of resuscitative mixtures as compared to a control crystalloid solution. The following protocol was used.

The catheters to be used in the experiment were flushed, and the rats were randomized to allow for three study arms. The rats were anesthetized and observed for at least 5 minutes prior to intervention. At t=0 minutes, blood began to be drawn from the carotid catheter to remove 40% of the animal's total blood volume to ˜45 mmHg MAP. At t=10 minutes the test resuscitative mixture or control solution was administered through the jugular catheter at a rate of lml/min. The test subject was continuously monitored (heart rate, pO₂ and MAP) every 5 minutes for 120 minutes. After 120 minutes, the test subject was euthanized.

The following resuscitative mixtures and/or controls were tested:
A1=the mixture of Table 1, col. 1, in solid form, +Sterile water
A2=0.9% Saline Solution
A3=the mixture of Table 1, col. 1, in solid form, +Plasma
A4=Thrombosomes+Plasma
A5=Thrombosomes+Plasma+the mixture of Table 1, col. 1, in solid form (combination of A3 and Thrombosomes),

The three study arms were as follows:

| Study Arm | | | | |
|---|---|---|---|---|
| (or Group) | Dose | Test Mixture | Control | Rats per study |
| 1 | 5 mL/kg | A1 | A2 | Total - 8 Rats |
| | | (Rats - 4) | (Rats - 4) | |
| 2 | 2.5 mL/kg | A1 | A2 | Total - 20 Rats |
| | | A3 | (Rats - 4) | |
| | | A4 | | |
| | | A5 | | |
| | | (Rats - 4 × 4 = | | |
| | | 16) | | |
| 3 | 1.25 mL/kg | A1 | A2 | Total - 8 Rats |
| | | (Rats - 4) | (Rats - 4) | |

The results were as follows.

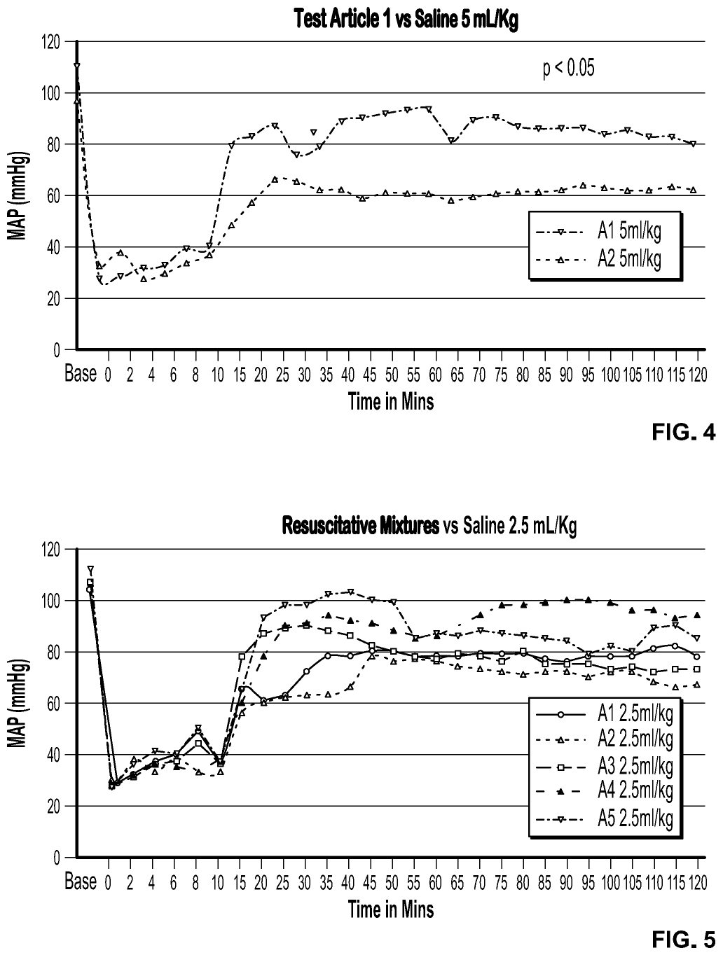

Group 1: A comparison of A1 (the mixture of Table 1, col. 1, in solid form+Sterile water) vs. A2 (0.9% Saline Solution) at 5 mL//kg is shown in FIG. 4. As can be seen from FIG. 4, the mixture of Table 1, col. 1, maintained a higher MAP than Saline at 5 ml/Kg.
Group 2: The following resuscitative mixtures and/or controls were tested:
A1=Mixture of Table 1, col. 1 (Solid)+Sterile water
A2=0.9% Saline Solution
A3=Mixture of Table 1, col. 1 (Solid)+Plasma
A4=Thrombosomes+Plasma
A5=Thrombosomes+Plasma+Mixture of Table 1, col. 1 (Solid) (Note: A5 is a combination of A3 and Thrombosomes)
   A comparison of all mixtures A1 (the mixture of Table 1, col. 1, in solid form, +Sterile water), A3 (the mixture of Table 1, col. 1, in solid form, +Plasma), A4 (Thrombosomes+Plasma), and A5 (Thrombosomes+Plasma+the mixture of Table 1, col. 1, in solid form, =combination of A3 and Thorombosomes), and of control A2 (0.9% Saline Solution), at 2.5 mL/kg is shown in FIG. 5.

As shown in FIG. 5, there is no substantive difference between A1 and A3, that is, between the mixture of Table 1, col. 1, in solid form, rehydrated with sterile water and the mixture of Table 1, col. 1, in solid form, rehydrated with plasma. FIG. 5 also shows that the mixture of Table 1, col. 1, in solid form, maintained a higher MAP than Saline at 2.5 ml/Kg. The difference in MAP values was less than the difference in MAP values between the mixture of Table 1, col. 1, in solid form, and Saline at 5 ml/Kg (see FIG. 4, above).

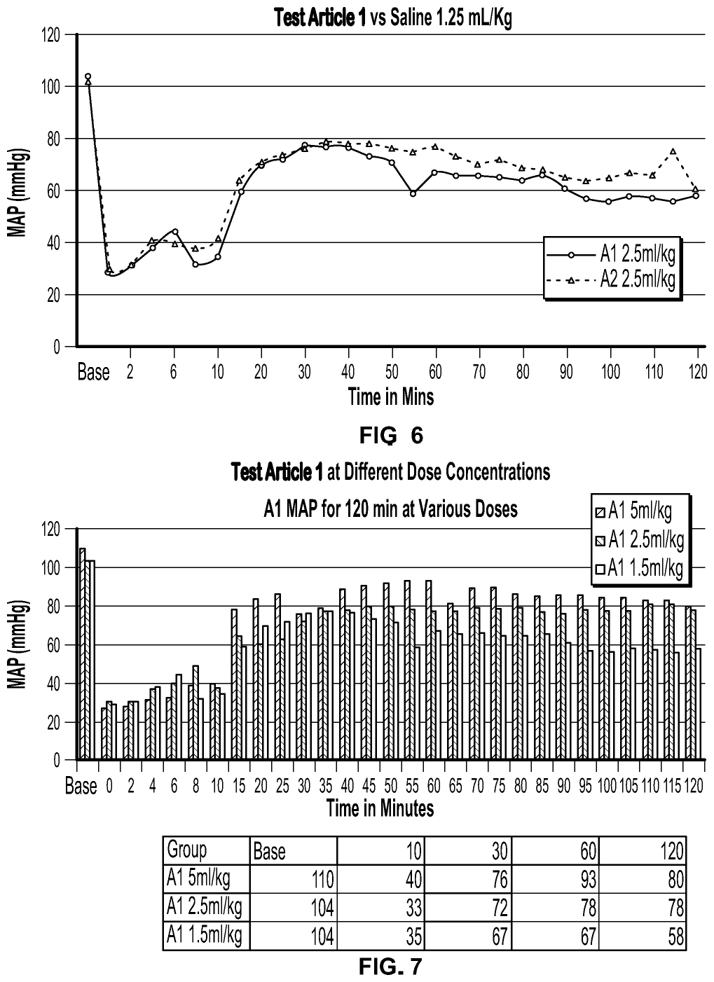

Inclusion of thrombosomes increased MAP, as shown, for example, by comparison of A3 and A5.
Group 3: A comparison of mixtures A1 (the mixture of Table 1, col. 1, in solid form, +Sterile water) and A2 (0.9% Saline Solution), at 1.25 mL/kg is shown in FIG. 6.
As shown in FIG. 6, saline maintained a similar or slightly higher MAP than the mixture of Table 1, col. 1, in solid form, at 1.25 ml/Kg.

Comparison of groups 1, 2 and 3 indicates that the mixture of Table 1, col. 1, in solid form, performs better compared to Saline as concentration increases from 1.25 mL/Kg to 5 mL/Kg. This is also borne out by the plot of FIG. 7, showing an increase in MAP at increasing concentrations of the mixture of Table 1, col. 1, in solid form, at most time points following administration.

### Exemplary Embodiments

1. A composition of matter comprising:
dried blood plasma substantially free of blood cell components; and
   a) a resuscitative mixture comprising at least one saccharide, at least one salt, and at least one high molecular weight, non-ionic, hydrophilic polymer,
   b) platelets or platelet-derived material,
   or
   c) both a) and b).
2. The composition of matter of claim 1, further comprising an aqueous liquid.
3. The composition of matter of claim 2, wherein the aqueous liquid is water or saline.
4. The composition of matter of claim 1, 2 or 3, wherein the resuscitative mixture further comprises an aqueous liquid.
5. The composition of matter of claim 4, wherein the aqueous liquid is water or saline.
6. The composition of matter of any one of claims 1 to 5, wherein, following rehydration, the platelets, platelet-derived material, or both, further contain an aqueous liquid.
7. The composition of matter of any one of claims 1 to 6, wherein the composition of matter does not comprise trehalose.
8. A composition of matter comprising:
   blood plasma rehydrated with an aqueous liquid and substantially free of blood cell components; and
   a) a resuscitative mixture comprising at least one saccharide, at least one salt, and at least one high molecular weight, non-ionic, hydrophilic polymer,
   b) platelets or platelet-derived material,
   or
   c) both a) and b);
   wherein the composition of matter comprises sufficient blood plasma, saccharide(s), salt(s), and high molecular weight, non-ionic, hydrophilic polymer(s) to provide a resuscitative effect when administered to a subject in need of blood resuscitation.
9. The composition of matter of claim 8, wherein the blood plasma has coagulation factor levels showing all individual factors at 40 international units (IU) or greater.
10. The composition of matter of any one of the preceding claims, wherein the at least one high molecular weight, non-ionic, hydrophilic polymer of the resuscitative mixture is a copolymer of sucrose and epichlorohydrin.
11. The composition of claim 10, wherein the copolymer is polysucrose.
12. The composition of matter of any one of the preceding claims, further comprising an additive for preservation of coagulation function.
13. The composition of claim 12, wherein the additive for preservation of coagulation function is L-Carnitine, Propionyl L-Carnitine, Taurine, Glycerophosphocholine (GCP) or Trehalose.
14. The composition of claim 12, wherein the additive for preservation of coagulation function comprises.taurine.
15. A composition comprising two or more of
   a. platelets, platelet-derived material, or both,
   b. plasma, or
   c. a mixture comprising a buffer, at least one saccharide, at least one salt, and at least one high molecular weight, non-ionic, hydrophilic polymer;
   wherein at least one of a), b), or c) is freeze-dried.
16. The composition of claim 15, comprising a., b. and c.
   A method for treating hypovolemia in a subject, comprising administering to the subject a composition of any one of claims 1 to 16.
17. The composition of claim 1, comprising the platelets or platelet-derived material, and the plasma.
18. The composition of claim 17, wherein the platelets are dried.
19. The composition of claim 17 or claim 18, wherein the platelets are freeze-dried.
20. The composition of claim 17, wherein the platelet-derived material is dried.
21. The composition of claim 17 or claim 20, wherein the platelet-derived material is freeze-dried.
22. The composition of any one of claims 17-21, wherein the composition is aqueous.
23. The composition of any one of claims 17-22, wherein the composition does not comprise a mixture comprising at least one salt and at least one high molecular weight, non-ionic, hydrophilic polymer.
24. The composition of any one of claims 17-22, wherein the composition does not comprise a mixture, wherein the mixture comprises a buffer, at least one saccharide, at least one salt, and at least one high molecular weight, non-ionic, hydrophilic polymer.
25. The composition of any one of claims 17-24, wherein the plasma is present in an amount from about 30% to about 80% wt/wt.
26. The composition of any one of claims 17-25, wherein the plasma is present in an amount from about 40% to about 70% wt/wt.
27. The composition of any one of claims 17-26, wherein the platelets or platelet-derived material are present in a concentration of about 10,000 platelets/μL to about 2,000,000 platelets/μL.
28. The composition of any one of claims 17-27, wherein the platelets or platelet-derived material are present in a concentration of about 100,000 platelets/μL to about 1,000,000 platelets/μL.
29. A method of treating hypovolemia in a subject, comprising administering to the subject the composition of claim 17.
30. The method of claim 29, wherein the platelets are dried.
31. The method of claim 29 or claim 30, wherein the platelets are freeze-dried.
32. The method of claim 29, wherein the platelet-derived material is dried.
33. The method of claim 29 or claim 32, wherein the platelet-derived material is freeze-dried. 34. The method of any one of claims 29-33, wherein the composition is aqueous.
35. The method of any one of claims 29-34, wherein the composition does not comprise a mixture comprising at least one salt and at least one high molecular weight, non-ionic, hydrophilic polymer.
36. The method of any one of claims 29-35, wherein the composition does not comprise a mixture, wherein the mixture comprises a buffer, at least one saccharide, at least one salt, and at least one high molecular weight, non-ionic, hydrophilic polymer.
37. A composition comprising:
   platelets or platelet-derived material; and
   a mixture comprising at least one salt and at least one high molecular weight, non-ionic, hydrophilic polymer;
   wherein the composition is a blood-infusible composition.
38. A method of treating hypovolemia in a subject, comprising administering to the subject a composition comprising the composition of claim 37.
39. The method of claim 38, wherein the mixture further comprises a buffer, at least one saccharide, or both.
40. The method of claim 38 or claim 39, wherein the platelets are dried.
41. The method of claim 40, wherein the platelets are freeze-dried.
42. The method of any one of claims 37-41, wherein the mixture is dried.
43. The method of any one of claims 37-40 and claim 42, wherein the platelets are dried and the mixture is dried.
44. The method of any one of claims 37, 38 and claim 42, wherein the platelet-derived material is dried.
45. The method of claim 44, wherein the platelet-derived material is freeze-dried.
46. The method of any one of claims 37, 38, 42, and 44, wherein the platelet-derived material is dried and the mixture is dried.
47. The method of any one of claims 37-46, wherein the mixture is Reovol™.
48. The method of any one of claims 37-47, wherein the composition is aqueous.
49. The method of any one of claims 37-48, wherein the composition does not comprise plasma.
50. The composition of claim 1, comprising the plasma; and
   the mixture comprising at least one salt and at least one high molecular weight, non-ionic, hydrophilic polymer.
51. The composition of claim 50, wherein the mixture further comprises a buffer, at least one saccharide, or both.
52. The composition of claim 50 or claim 51, wherein the plasma is freeze-dried.
53. The composition of any one of claims 50-52, wherein the mixture is dried.
54. The composition of any one of claims 50-53, wherein the mixture is Reovol™.
55. The composition of any one of claims 50-54, wherein the composition is aqueous.
56. The composition of any one of claims 50-55, wherein the plasma is present in an amount from about 30% to about 80% wt/wt.
57. The composition of any one of claims 50-56, wherein the plasma is present in an amount from about 40% to about 70% wt/wt.
58. The composition of any one of claims 50-57, wherein the mixture is present in an amount from about 20% to about 70% wt/wt.
59. The composition of any one of claims 50-58, wherein the mixture is present in an amount from about 30% to about 60% wt/wt.
60. The composition of any one of claims 50-59, wherein the composition does not comprise platelets or platelet-derived material.
61. A method of treating hypovolemia in a subject, comprising administering to the subject the composition of any one of claims 50-60.
62. The method of claim 61, wherein the mixture further comprises a buffer, at least one saccharide, or both.
63. The method of claim 61 or claim 62, wherein the mixture is dried.
64. The method of any one of claims 61-63, wherein the mixture is Reovol™.
65. The method of any one of claims 61-64, wherein the composition is aqueous.
66. The method of any one of claims 61-65, wherein the composition does not comprise platelets or platelet-derived material.
67. The composition of claim 1, comprising:
   platelets or platelet-derived material;
   plasma; and
   a mixture comprising at least one salt and at least one high molecular weight, non-ionic, hydrophilic polymer.
68. The composition of claim 67, wherein the mixture further comprises a buffer, at least one saccharide, or both.
69. The composition of claim 67 and claim 68, wherein the platelets are dried.
70. The composition of any one of claims 67-69, wherein the platelets are freeze-dried.
71. The composition of claim 67, wherein the platelet-derived material is dried.
72. The composition of claim 67 or claim 71, wherein the platelet-derived material is freeze-dried.
73. The composition of any one of claims 67-72, wherein the mixture is dried.
74. The composition of any one of claims 67-73, wherein the mixture is freeze-dried.
75. The composition of any one of claims 67-70, and 73, wherein the platelets are dried and the mixture is dried.
76. The composition of any one of claims 67, 71-72, and 73-74, wherein the platelet-derived material is dried, and the mixture is dried.
77. The composition of any one of claims 67-76, wherein the mixture is Reovol™.
78. The composition of any one of claims 67-77, wherein the composition is aqueous.
79. The composition of any one of claims 67-78, wherein the plasma is present in an amount from about 40% to about 60% wt/wt.
80. The composition of any one of claims 67-79, wherein the plasma is present in an amount from about 30% to about 50% wt/wt.
81. The composition of any one of claims 67-80, wherein the platelets or platelet-derived material are present in a concentration of about 10,000 platelets/μL to about 800,000 platelets/μL.
82. The composition of any one of claims 67-81, wherein the platelets or platelet-derived material are present in a concentration of about 100,000 platelets/μL to about 800,000 platelets/μL.
83. The composition of any one of claims 67-82, wherein the mixture is present in an amount from about 20% to about 40% wt/wt.
84. The composition of any one of claims 67-83, wherein the mixture is present in an amount from about 30% to about 40% wt/wt.
85. A method of treating hypovolemia in a subject, comprising administering to the subject the compositions of any one of claims 67-84.
86. The method of claim 85, wherein the mixture further comprises a buffer, at least one saccharide, or both.
87. The method of claim 85 or claim 86, wherein the platelets are dried.
88. The method of any one of claims 85-87, wherein the platelets are freeze-dried.
89. The method of claim 85 or claim 86, wherein the platelet-derived material is dried.
90. The method of claim any one of claims 85, 86, and 89, wherein the platelet-derived material is freeze-dried.
91. The method of any one of claims 85-90, wherein the mixture is dried.
92. The method of any one of claims 85-90, wherein the mixture is freeze-dried.
93. The method of any one of claims 85-87, and 91, wherein the platelets are dried and the mixture is dried.
94. The method of any one of claims 85, 86, 89, and 91, wherein the platelet-derived material is dried and the mixture is dried.
95. The method of any one of claims 85-94, wherein the mixture is Reovol™.
96. The method of any one of claims 85-95, wherein the composition is aqueous.
97. A composition of matter comprising:
   a) blood plasma substantially free of blood cell components;
   b) a resuscitative mixture comprising at least one saccharide, at least one salt, and at least one high molecular weight, non-ionic, hydrophilic polymer, and
   c) platelets or platelet-derived material,
   wherein each of a), b), and c) is dried or rehydrated; and
   wherein the composition of matter comprises sufficient blood plasma, saccharide(s), salt(s), and high molecular weight, non-ionic, hydrophilic polymer(s) to provide a resuscitative effect when administered to a subject in need of blood resuscitation.
98. The composition of matter of claim 97, wherein the blood plasma is blood plasma rehydrated with an aqueous liquid.
99. The composition of matter of claim 97, wherein the blood plasma is dried.
100. The composition of matter of claim 97, wherein the resuscitative mixture is resuscitative mixture rehydrated with an aqueous liquid.
101. The composition of matter of claim 97, wherein the resuscitative mixture is dried.
102. The composition of matter of claim 97, wherein the platelets or the platelet-derived material is rehydrated with an aqueous liquid.
103. The composition of matter of claim 97, wherein the platelets or the platelet-derived material is dried
104. The composition of matter of one of claims 98, 100, and 102, wherein the aqueous liquid is water.
105. The composition of matter of claims 98, 100, 102, and 104, wherein the aqueous liquid is sterile water.
106. The composition of matter of claim 97, wherein the blood plasma has coagulation factor levels showing all individual factors at 40 international units (IU) or greater.
107. The composition of matter of claim 97, wherein the composition of matter has coagulation factor that include only Factors VII, VIII and IX.
108. A composition of matter comprising:
   a) red blood cells or red blood cell substitutes,
   b) a resuscitative mixture comprising at least one saccharide, at least one salt, and at least one high molecular weight, non-ionic, hydrophilic polymer, and
   c) platelets or platelet-derived material,
   wherein each of a), b), and c) is dried or rehydrated.
109. A composition of matter comprising:
   a) red blood cells or red blood cell substitutes,
   b) dried blood plasma,
   c) a resuscitative mixture comprising at least one saccharide, at least one salt, and at least one high molecular weight, non-ionic, hydrophilic polymer, and
   d) platelets or platelet-derived material,
   wherein each of a), b), c) and d) is dried or rehydrated.

It will be apparent to those skilled in the art that various modifications and variations can be made in the practice of the present invention without departing from the scope or spirit of the invention. Certain embodiments will be apparent to those skilled in the art from consideration of the specification. It is intended that the specification and examples be considered as exemplary only, with a true scope and spirit of the invention being indicated by the following claims.

1. A composition of matter comprising:
dried blood plasma substantially free of blood cell components; and
a) a resuscitative mixture comprising at least one saccharide, at least one salt, and at least one high molecular weight, non-ionic, hydrophilic polymer,
b) platelets or platelet-derived material,
or
c) both a) and b).

2. The composition of matter of claim 1, further comprising an aqueous liquid.

3. The composition of matter of claim 1, wherein the aqueous liquid is water or saline.

4. The composition of matter of claim 1, wherein the resuscitative mixture further comprises an aqueous liquid.

5. The composition of matter of claim 4, wherein the aqueous liquid is water or saline.

6. The composition of matter of claim 1, wherein, following rehydration, the platelets, platelet-derived material, or both, further contain an aqueous liquid.

7. The composition of matter of claim 1, wherein the composition of matter does not comprise trehalose.

8. A composition of matter comprising:
blood plasma rehydrated with an aqueous liquid and substantially free of blood cell components; and
a) a resuscitative mixture comprising at least one saccharide, at least one salt, and at least one high molecular weight, non-ionic, hydrophilic polymer,
b) platelets or platelet-derived material,
or
c) both a) and b);
wherein the composition of matter comprises sufficient blood plasma, saccharide(s), salt(s), and high molecular weight, non-ionic, hydrophilic polymer(s) to provide a resuscitative effect when administered to a subject in need of blood resuscitation.

9. The composition of matter of claim 8, wherein the blood plasma has coagulation factor levels showing all individual factors at 40 international units (IU) or greater.

10. The composition of matter of claim 9, wherein the at least one high molecular weight, non-ionic, hydrophilic polymer of the resuscitative mixture is a copolymer of sucrose and epichlorohydrin.

11. The composition of matter of claim 10, wherein the copolymer is polysucrose.

12. The composition of matter of claim 8, further comprising an additive for preservation of coagulation function.

13. The composition of matter of claim 12, wherein the additive for preservation of coagulation function is L-Carnitine, Propionyl L-Carnitine, Taurine, Glycerophosphocholine (GCP) or Trehalose.

14. The composition of matter of claim 12, wherein the additive for preservation of coagulation function comprises.taurine.

15. A composition comprising two or more of
a. platelets, platelet-derived material, or both,
b. plasma, or
c. a mixture comprising a buffer, at least one saccharide, at least one salt, and at least one high molecular weight, non-ionic, hydrophilic polymer;
wherein at least one of a), b), or c) is freeze-dried.

16. The composition of claim 15, comprising a, b. and c.

17. A method for treating hypovolemia in a subject, comprising administering to the subject a composition of claim 16.

18. The composition of claim 17, wherein the platelets are dried.

19. The composition of claim 17, wherein the platelets are freeze-dried.

20. The composition of claim 17, wherein the platelet-derived material is dried.

21. The composition of claim 17, wherein the platelet-derived material is freeze-dried.
